SEQUENCE LISTING (27 sequences)

SEQ ID NO: 1 - DNA, 21 bp, primer
tcccaggtgcagctggtgcag

SEQ ID NO: 2 - DNA, 19 bp, primer
ctgagctcacggtcaccag

SEQ ID NO: 3 - DNA, 21 bp, primer
tccgacatcctgctgacccag

SEQ ID NO: 4 - DNA, 19 bp, primer
gtttgatctccaccttggt

SEQ ID NO: 5 - DNA, 85 bp, primer
ccctggtgaccgtgagctcaggtggcggtggctcgggcggtggtgggtcgggtggcggcggatctgacatcctgctgacccagag

SEQ ID NO: 6 - DNA, 15 bp, primer
ggggsggggsggggs

SEQ ID NO: 7 - DNA, 35 bp, primer
gcggcccagccggcccaggtgcagctggtgcagag

SEQ ID NO: 8 - DNA, 34 bp, primer
ctgcggccgctttgatctccaccttggtgccctg

SEQ ID NO: 9 - DNA, 38 bp, primer
tcccaagctttcaccatgcaggtgcagctggtgcagag

SEQ ID NO: 10 - DNA, 33 bp, primer
cccgctcgagtcatttgatctccaccttggtgc

SEQ ID NO: 11 - DNA, 34 bp, primer
tcccagatctaagcttgccgccaccatggactgg

SEQ ID NO: 12 - DNA, 22 bp, primer
gctgatctcgcccacccactcc

SEQ ID NO: 13 - DNA, 30 bp, primer
cccgagatctcgagtcatttgatctccacc

SEQ ID NO: 14 - DNA, 27 bp, primer
ggagatctccacagtccctgaacacac

SEQ ID NO: 15 - DNA, 21 bp, primer
tcatttacccggagacaggga

SEQ ID NO: 16 - DNA, 21 bp, primer
ctaacactctcccctgttgaa

SEQ ID NO: 17 - DNA, 27 bp, primer
tgaagaaagcttgccgccaccatggag

SEQ ID NO: 18 - DNA, 29 bp, primer
gcatccgctcgtttgatctccaccttggt

SEQ ID NO: 19 - DNA, 38 bp, primer
cggaattcgagcggatgctgcaccaactgtatcgatct

SEQ ID NO: 20 - DNA, 40 bp, primer
gctctagagctaacactcattcctgttgaagctcttgaca

SEQ ID NO: 21 - DNA, 21 bp, primer
tgagctcacggtcaccagggt

SEQ ID NO: 22 - DNA, 369 bp, human/murine
  CDS (1)...(366)
caggtgcagctggtgcagagcggcgccgaggtgaagaagcccggcgccagcgtgaaggtgagctgcaaggccagcggctacaccttcagcatgtactggctggagtgggtgaggcaggcccccggccacggcctggagtgggtgggcgagatcagccccggcaccttcaccaccaactacaacgagaagttcaaggccagggccaccttcaccgccgacaccagcaccaacaccgcctacatggagctgagcagcctgaggagcgaggacaccgccgtgtactactgcgccaggttcagccacttcagcggcagcaactacgactacttcgactactggggccagggcaccctggtgaccgtgagctca

SEQ ID NO: 23 - protein, 122 aa, human/murine
QVQLVQSGAEVKKPGASVKVSCKASGYTFSMYWLEWVRQAPGHGLEWVGEISPGTFTTNYNEKFKARATFTADTSTNTAYMELSSLRSEDTAVYYCARFSHFSGSNYDYFDYWGQGTLVTVS

SEQ ID NO: 24 - DNA, 321 bp, human/murine
  CDS (1)...(321)
gacatcctgctgacccagagccccggcaccctgagcctgagccccggcgagagggccaccctgagctgcagggccagccagagcatcggcaccaacatccactggtaccagcagaagcccggccaggcccccaggctgctgatcaagtacgccagcgagagcatcagcggcatccccagcaggttcagcggcagcggcagcggcaccgacttcaccctgaccatcagcaggctggagcccgaggacttcgccatgtactactgccagcagagcgacagctggcccaccaccttcggccagggcaccaaggtggagatcaaa

SEQ ID NO: 25 - protein, 107 aa, human/murine
DILLTQSPGTLSLSPGERATLSCRASQSIGTNIHWYQQKPGQAPRLLIKYASESISGIPSRFSGSGSGTDFTLTISRLEPEDFAMYYCQQSDSWPTTFGQGTKVEIK

SEQ ID NO: 26 - DNA, 735 bp, human/murine
  CDS (1)...(735)
caggtgcagctggtgcagagcggcgccgaggtgaagaagcccggcgccagcgtgaaggtgagctgcaaggccagcggctacaccttcagcatgtactggctggagtgggtgaggcaggcccccggccacggcctggagtgggtgggcgagatcagccccggcaccttcaccaccaactacaacgagaagttcaaggccagggccaccttcaccgccgacaccagcaccaacaccgcctacatggagctgagcagcctgaggagcgaggacaccgccgtgtactactgcgccaggttcagccacttcagcggcagcaactacgactacttcgactactggggccagggcaccctggtgaccgtgagctcaggtggcggtggctcgggcggtggtgggtcgggtggcggcggatctgacatcctgctgacccagagccccggcaccctgagcctgagccccggcgagagggccaccctgagctgcagggccagccagagcatcggcaccaacatccactggtaccagcagaagcccggccaggcccccaggctgctgatcaagtacgccagcgagagcatcagcggcatccccagcaggttcagcggcagcggcagcggcaccgacttcaccctgaccatcagcaggctggagcccgaggacttcgccatgtactactgccagcagagcgacagctggcccaccaccttcggccagggcaccaaggtggagatcaaa

SEQ ID NO: 27 - protein, 245 aa, human/murine
QVQLVQSGAEVKKPGASVKVSCKASGYTFSMYWLEWVRQAPGHGLEWVGEISPGTFTTNYNEKFKARATFTADTSTNTAYMELSSLRSEDTAVYYCARFSHFSGSNYDYFDYWGQGTLVTVSSGGGGSGGGGSGGGGSDILLTQSPGTLSLSPGERATLSCRASQSIGTNIHWYQQKPGQAPRLLIKYASESISGIPSRFSGSGSGTDFTLTISRLEPEDFAMYYCQQSDSWPTTFGQGTKVEIK

(19) United States
(12) Patent Application Publication
(10) Pub. No.: US 2002/0173477 A1
(43) Pub. Date: Nov. 21, 2002

(54) ANTI-IgE gene therapy

(76) Inventors: Ruey S. Liou, Sugarland, TX (US); David Thomas, Houston, TX (US)

Correspondence Address: TANOX, INC., 10301 STELLA LINK, HOUSTON, TX, 77025, US

(21) Appl. No.: 09/991,470

(22) Filed: Nov. 20, 2001

Related U.S. Application Data
(63) Continuation of application No. 09/397,569, filed on Sep. 16, 1999, now abandoned.
(60) Provisional application No. 60/100,639, filed on Sep. 16, 1998.

(51) Int. Cl. A61K 48/00; C12N 5/06

(52) U.S. Cl. 514/44; 435/69.1; 435/326

(57) ABSTRACT

Disclosed are gene constructs of anti-IgE antibodies or fragments thereof for therapy for allergic diseases. Upon introduction into suitable host, anti-IgE antibody gene constructs will direct the synthesis of an antibody (or its fragments) capable of binding to free IgE in serum but not binding to IgE bound to the high affinity receptor (FcεRI), or not binding to IgE bound to both the high affinity receptor and the low affinity receptor (FcεRII or CD23). The antibody (or fragments) which are synthesized may also inhibit IgE binding to the high affinity receptor or the low affinity receptor, or to both.

### RELATED APPLICATION

This application claims priority to U.S. Provisional Application Ser. No. 60/100,639 filed on Sep. 16,1998; and to U.S. application Ser. No. 09/397,569, filed on Sep. 16,1999.

### BACKGROUND OF THE INVENTION

IgE binds to the α chain of the high affinity IgE Fc receptor (FcεRI) present on mast cells and basophils, and to the low affinity receptor (FcεR2, CD23), present on monocytes/macrophages, lymphocytes, and dendritic cells. Cross-linking of IgE molecules bound to mast cells by allergens aggregates the underlying FcεRI receptors and triggers a series of biochemical events that result in the activation of these cells and release of preformed and newly generated vasoactive and bronchoconstrictive substances, leading to the immediate hypersensitivity type responses, such as the early phase of airway obstruction, allergic rhinitis, and other IgE-mediated allergic diseases. IgE cross-linking may also trigger the release of cytokines within the mast cells, including IL-4, IL-5, IL-6, and TNF-α, suggesting an important role for IgE in the late phase of airway obstruction and the associated increase in bronchial hyperresponsiveness. Moreover, by acting directly through FcεRII, IgE may also play a central role in the induction of a Th2-type response, and form part of a positive feedback loop leading to further increases in IgE and causing airway eosinophilia. Thus, therapy that can interfere with IgE binding to high affinity receptors, or high and low-affinity receptors, should inhibit these biochemical events and reduce the early and late phase airway responses through blocking of mast cell degranulation.

Murine anti-IgE monoclonal antibodies, which interfere in the binding to both the high and low affinity receptors have been generated. These antibodies bind the high and low-affinity receptor-binding portions of human IgE located in the Cε3 domain. They bind to circulating IgE and IgE expressed on the surface B cells (membrane-bound IgE). They do not bind to IgE already bound to the FcεRI on mast cells and basophils or FcεRII on lymphocytes and other cells bearing the receptor. Consequently, they do not activate these cells and trigger the release of mediators. Chimeric versions of the antibody, which consist of the heavy and light chain variable regions of the murine parent antibody and the heavy and light chain constant regions of the human γ1 and κ antibody isotypes, or a humanized version of the antibody, which retains the complementarity determining regions (CDRs) of the heavy and light chain variable regions with the majority of the remainder of the antibody (except for some portions of the framework regions) replaced with the heavy and light chain of the human γ1 and κ antibody isotypes, were shown to retain essentially identical antigen binding specificity and affinity. These antibodies have demonstrated their anticipated activity in neutralizing circulating IgE and at the same time ameliorating allergic symptoms in atopic patients in human clinical studies.

The anticipated treatment regimen for anti-IgE antibodies is subcutaneous injection at 3-4 weeks intervals during pollen season for allergic rhinitis, and year round for allergic asthma. Gene therapy allows administering the gene constructs for the anti-IgE antibody or its fragments into appropriate tissue sites for a more sustained expression of the antibody, resulting in better control of the serum IgE levels.

### SUMMARY OF THE INVENTION

The invention includes gene constructs of anti-IgE antibodies or fragments thereof for therapy. Upon introduction into suitable host, anti-IgE antibody gene constructs will direct the synthesis of an antibody (or its fragments) capable of binding to free IgE in serum but not binding to IgE bound to the high affinity receptor (FcεRI), or not binding to IgE bound to both the high affinity receptor and the low affinity receptor (FcεRII or CD23). The antibodies (or fragments) which are synthesized may also inhibit IgE binding to the high affinity receptor or the low affinity receptor, or both.

These constructs include genes for whole antibody molecules as well as modified or derived forms thereof, including immunoglobulin fragments like Fab, single chain Fv (scFv) and F(ab′)₂. The anti-IgE antibodies and fragments can be animal-derived, human-mouse chimeric, humanized, DeImmunized™ or fully from human. The gene construct can be introduced into a host with conventional gene therapy techniques, including as naked DNA, DNA incorporated in liposomes, DNA conjugated to lipids or to lipid derivatives or via suitable plasmids or recombinant viral vectors.

Humanized anti-IgE genes may be incorporated into a recombinant adenovirus vector as an independent transcriptional unit, and packaged into infectious virus particles. Upon infection of host, the recombinant adenovirus will direct the production of either intact anti-IgE antibody or an scFv fragment in serum, which will bind free circulating IgE, resulting in the reduction of free serum IgE. The binding of the antibody or fragment to IgE-bearing B cells may lower IgE levels by down-regulating IgE production by these B cells.

### BRIEF DESCRIPTION OF THE SEQUENCE LISTING

SEQ ID NOS:1 to 21 are various primers, used in the manner described below.

SEQ ID NO:22 is the DNA sequence of the VH region of the humanized antibody Hu-901.

SEQ ID NO:23 is the amino acid sequence of the DNA of SEQ ID NO:22.

SEQ ID NO:24 is the DNA sequence of the Vκ region of the humanized antibody Hu-901.

SEQ ID NO:25 is the amino acid sequence of the DNA sequence of SEQ ID NO:24.

SEQ ID NO:26 is the DNA sequence of the scFv fragment of the humanized antibody Hu-901.

SEQ ID NO:27 is the amino acid sequence of the DNA sequence of SEQ ID NO:26.

### BRIEF DESCRIPTIONS OF THE DRAWINGS

FIG. 1 shows three schematic diagrams of the recombinant adenovirus constructs for the scFv fragment of Hu-901 (top), Hu-901 (middle), and Hu-901 with a murine constant region (lower). LITR refers to adenovirus type 5 (Ad5) 5′ inverted terminal repeats along with the Ad5 origin of replication, the Ad5 encapsidation signal, and the E1a enhancer. RITR refers to adenovirus type 5 (Ad5) 3′ inverted terminal repeats. PhCMV is a promoter sequence derived from human cytomegloavirus; pA is a polyadenylation signal from SV40; E1 and E3 are the early region genes of adenovirus virus; and GFP is the green fluorescence protein.

FIG. 2 shows the expression of Hu-901 (mCγ2a,κ) in FVB mice infected with different doses of AdHu-901(mCγ2a,κ) virus, where 1×10⁹ pfu/mouse denotes each mouse infected with 1×10⁹ plaque forming units of the recombinant adenovirus construct, and 5×10⁸ pfu/mouse is a mouse infected with 5×10⁸ plaque forming units of the recombinant adenovirus construct.

FIG. 3 shows the expression of scFv_{Hu-901} in FVB mice infected with different doses of AdscFv_{Hu-901} virus.

FIG. 4 shows the expression of scFv_{Hu-901} in FVB mice infected with the same dose of AdscFv_{Hu-901} virus.

FIGS. 5A to 5C show the effects of the expressed Hu-901(mCγ2a,κ) and scFv_{Hu-901} on the free circulating human Cε-containing IgE in Hu-IgE transgenic mice infected with AdHu-901(mCγ2a,κ) and AdscFv_{Hu-901} viruses. FIG. 5A depicts the mean free circulating IgE from three untreated Hu-IgE transgenic mice; FIG. 5B depicts the effect of Hu-901 (mCγ2a,κ) on free IgE levels in 5 mice infected with AdHu-901(mCγ2a,κ) virus; and FIG. 5C depicts the effect of scFv_{Hu-901} on free IgE levels in 5 mice infected with AdscFv_{Hu-901} virus.

### MAKING AND USING THE INVENTION

Producing the Antibody Gene Constructs of the Invention

The anti-IgE antibody gene constructs described herein may encode antibodies that target a specific epitope on IgE that overlaps with IgE binding epitopes to both high and low-affinity receptors, FcεRI and FcεRII, respectively. Exemplary anti-IgE antibody include the monoclonal antibodies produced by hybridoma TES-C21, and its chimeric mouse-human form, produced by transfectoma lines TESC-2 (as described in International Application No. W092/17207). A humanized version of TES-C21 (designated Hu-901) is described in Australian Patent No. 675449. Gene constructs encoding DeImmunized™ and human antibodies with desired target specificity against IgE can also be prepared using conventional techniques.

To prepare the gene constructs, the genes encoding the heavy and light chain of the chimeric antibody (Hu-901) is obtained through RT-PCR using the RNA from the transfectoma cell line producing the chimeric antibody. The cell line is deposited in the American Type Culture Collection (ATCC), 10801 University Blvd., Manassas, Va., 10110, under Accession No. BRL 10706. After sequence confirmation, the cDNA fragments are separately ligated to an expression vector under the transcriptional control of a strong promoter, for example, human CMV promoter, the EF1 promoter or albumin promoter, and a polyadenylation signal site is provided either by the antibody DNA fragments or from the vector that contains the poly A site derived from SV40, β-globin gene or another appropriate source. Alternatively, the heavy and light chain genes can be placed in one plasmid construct either under separate promoter control or under one promoter in a dicistronic arrangement. The antibody gene fragments can also be placed under the control of proper promoters that allow the turning on and off of gene expression with appropriate exogeneous factors, such as steroids or metal ions. Gene constructs for a humanized anti-IgE antibody can be similarly prepared using RNA from transfectoma cells producing a humanized anti-IgE antibody. Examples include cell lines deposited in ATCC under the following Accession numbers: 11130, 11131, 11132, 11133. Alternatively, genomic DNA constructs containing exons, introns and immunoglobulin transcriptional regulatory sequences, promoters and enhancers can also be constructed. Gene constructs directing the expression of antibody fragments such as Fab, F(ab′)₂, single-chain Fv (scFv), can also be constructed by preparing the suitable gene segments encoding these antibody fragments which are ligated to suitably prepared vectors.

The gene constructs incorporated into the viral genome and subsequently packaged into suitable viral particles can allow a high efficiency gene delivery through viral infection. Exemplary viral vectors commonly used for genetic therapy include retrovirus vectors, adenovirus vectors and adeno-associated virus (AAV) vectors. The more recently developed viral vectors suitable for genetic therapy include lentivirus (HIV-1 or HIV-2 based vectors), and alphavirus vectors (based on Sindbis virus and Semliki Forest virus). Anti-IgE gene constructs can be incorporated into viral genomes of retroviruses, lentiviruses or AAV vectors by subcloning of the transcriptional units into appropriate cassette vectors containing necessary sequences for virus packing. Upon DNA transfection of the resulting constructs into appropriate packaging cell lines that produce viral components, the recombinant viral genomes can be properly packaged into viable viral particles.

To incorporate the anti-IgE gene constructs into an adenoviral viral genome, an additional step is generally taken. Since the adenoviral genome is approximately 36 Kbp long, it is not convenient to directly insert the anti-IgE gene into the genome through restriction endonuclease digestion and ligation. Instead, anti-IgE genes are inserted in a cassette vector such as pAvCvSv (Kobayashi K et al. (1996) J. Biol. Chem. 22:6852-60). The vector has a pBR322 backbone and contains adenovirus type 5 (Ad5) 5′ inverted terminal repeats (ITR), the Ad5 origin of replication, the Ad5 encapsidation signal, the E1a enhancer, multiple cloning sites, and Ad5 sequence from nucleotide positions 3328 to 6246, which serve as a homologous recombination fragment. The resulting plasmid is then co-transfected into an appropriate host cell line, such as 293 cells (Graham FL, J Smiley, W C Russell and R Nairn, (1977) J. Gen. Viro. 36:59-72), along with a DNA fragment containing the bulk of the adenoviral genome with deletions in certain vital regions, such as the E1 and E3 genes. Homologous recombination between two DNAs in overlapping regions would allow the generation of a recombinant viral genome harboring the anti-IgE genes. This recombinant genome will be subsequently packaged into viable infectious viral particles in the 293 host cells. Incorporation of anti-IgE genes into the genome of alphaviruses or other viruses with a large genome can be similarly carried out to generate recombinant virus.

These gene constructs can be prepared as plasmids, which can be delivered to host cells or tissues, either directly or as naked DNA, or as DNA incorporated in liposomes, conjugated with appropriate lipid components, or incorporated in viral vectors. They are preferably injected for administration. The gene constructs will be expected to direct the synthesis of anti-IgE or its fragments, which will gradually enter the blood stream to interact with IgE. The recombinant virus constructs can be administered into an individual with allergic diseases via intra-muscular, intravenous, or subcutaneous routes. The dosage can be determined by extrapolating from animal experiments or determined in human clinical trials.

### EXAMPLE 1

### Preparation of DNA Construct for the Expression of a scFv Fragment of Anti-IgE with Humanized V Regions.

A DNA construct for scFv without leader/signal peptide sequence for expression in mammalian cells was first prepared as follows. A polymerase chain reaction (PCR) was set up by using the pHCMV-V_{H3}-huC₁ plasmid DNA as the template, and oligonucleotides:

| | | |
|---|---|---|
| H3-5 | 5′-TCCCAGGTGCAGCTGGTGCAG-3′ | (SEQ ID NO:1); and |
| H3-3 | 5′-CTGAGCTCACGGTCACC-AG-3′ | (SEQ ID NO:2) |

as the 5′ and 3′ primers, respectively. A 380-bp DNA fragment of the Hu-901 heavy chain V gene, V_{H3}, was obtained. A 330-bp DNA fragment of the Hu-901 light chain V gene, V_{L1}, was obtained by PCR using oligonucleotides:

| | | |
|---|---|---|
| L1-5 | 5′-TCCGACATCCTGCTGACCCAG-3′ | (SEQ ID NO:3); and |
| L1-3 | 5′-GTTTGATCTCCACCTTGGT-3′ | (SEQ ID NO:4) |

as the 5′ and 3′ primers, respectively. The pHCMV-V_{L1}-huCκ plasmid DNA was used as the template in this PCR. The H3L1-LINK oligonucleotide:

(5′-CCCTGGTGACCGTGAG-CTCAGGTGGCGGTGGCTCGGGCGGTGGTGGGTCGGGTGG
CGGCGGATCTGACATCCTGCTGACCCAGAG-3′SEQ ID NO: 5)

was synthesized to contain the 3′ end of the V_{H3} exon, nucleotides encoding the GGGGSGGGGSGGGGS peptide (SEQ ID NO: 6), and the 5′ end of the V_{L1} exon. PCR products of the V_{H3} and V_{L1} DNA fragments, together with the H3L1-LINK (SEQ ID NO: 5) oligonucleotide were used in PCR under the condition of 94° C., 1 min; 63° C., 4 min, for 7 cycles. A second PCR was carried out using the above mixture as the template and oligonucleotides:

| | | |
|---|---|---|
| SFI-H3 | 5′-GCGGCCCAGCCGGCCCAGGTGCAGCTGGTGCAGAG-3′ | (SEQ ID NO:7); and |
| L1-NOT | 5′-CTGCGGCCGCTTTGATCTCCACCTTGGTGCCCTG | (SEQ ID NO: 8) |

as the primers under the conditions of 94° C., 1 min; 55° C., 2 min; 72° C., 2 min, for 30 cycles. The resulting 750-bp DNA fragments were digested with restriction enzymes SfiI and NotI and inserted into the pCANTAB5E phagemid vector. Sixteen out of 17 colonies were shown to contain the correct size inserts by PCR. One plasmid DNA was used as the template in PCR using oligonucleotides:

| | | |
|---|---|---|
| 5TES | 5′-TCCCAAGCTTTCACCAT-GCAGGTGCAGCTGGTGCAGAG-3′ | (SEQ ID NO:9); |
| and 3TES | 5′-CCCGCTCGAGTCATTTGATCTCCACCTTGGTGC-3′ | (SEQ ID NO:10) |

as the primers. The 750-bp DNA fragments were digested with restriction enzymes HindIII and XhoI and then inserted into the pcDNA3 plasmid to give pcDNA3-H3L1scFv.

A synthetic leader/signal peptide sequence was added to the leaderless scFv fragment according the process described below. A 240-bp DNA fragment containing the leader sequence and the 5′ end of the humanized Hu-901 V region gene (approximately to the end of FR2) was obtained by polymerase chain reaction (PCR) using oligonucleotide:

| | | |
|---|---|---|
| H3-5BH | 5′TCCCAGATCTAAGCTTGCCGCCACCATGGACTGG3′ | (SEQ ID NO: 11); and |
| H3-3S | 5′GCTGATCTCGCCCACCCACTCC3′ | (SEQ ID NO: 12) |

as PCR primers and plasmid pHCMV-V_{H3}hCγ1 as the template. This PCR product was subsequently mixed with the leaderless scFv DNA fragment, and allowed for annealing and sequence extension in the presence of AmpliTaq under the conditions of 94° C., 1 min.; 55° C., 2 min.; and 72° C., 1 min., for 25 cycles. The resulting DNA was used directly as a template for the amplification of full-length signal/leader peptide-containing scFv fragment in a PCR reaction using oligonucleotides:

| | | |
|---|---|---|
| H3-5BH | (SEQ ID NO: 11); and | tl,27 |
| LI-3BX | 5′CCCGAGATCTCGAGTCATTTGATCTCCACC | (SEQ ID |
| | | NO:13) |

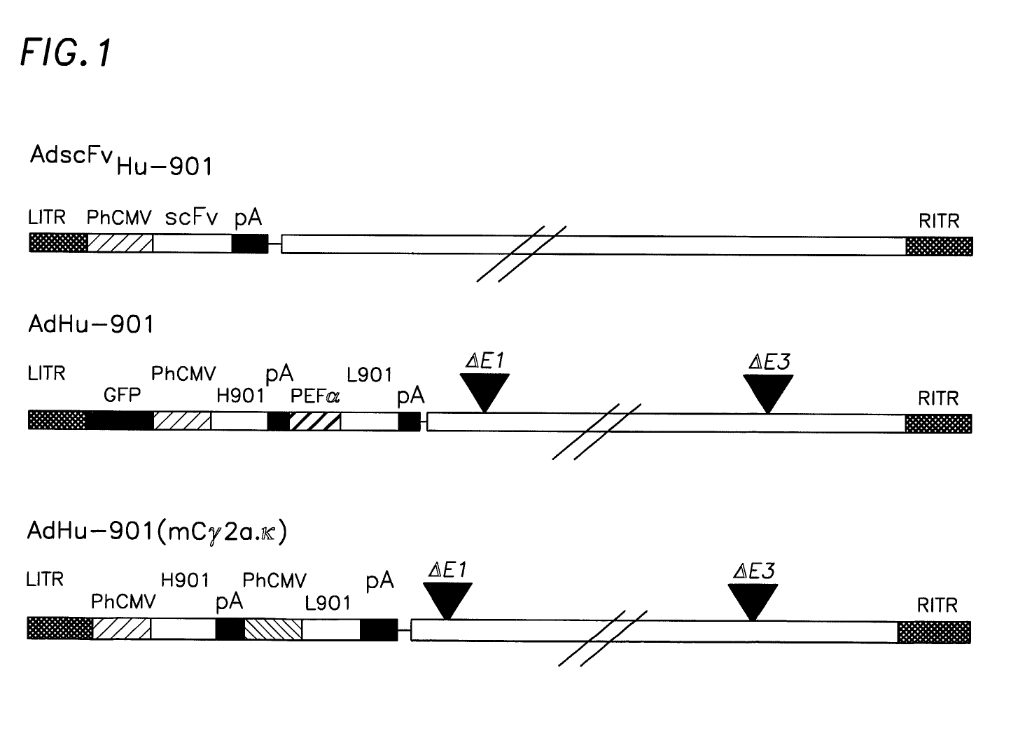

as primers. The full-length scFv DNA thus obtained was ligated to vector pCR® Blunt (Invitrogen, Carlsbad, Calif.) according to the conditions recommended by the supplier and transformed to TOP10 ONE SHOT™ competent cells. Six transformants were randomly selected and the plasmids were purified for sequence confirmation. DNA sequence determination was performed with ABI PRISM™ BIG DYE™ Terminator Cycle Sequencing Reaction Kit and analyzed by ABI PRISM™ 300 Genetic Analyzer (Perkin Elmer, Foster City, Calif.). The plasmid DNA from one clone that contained the expected sequence encoding scFv was digested by EcoRI, treated with DNA polymerase Klenow fragment, and the scFv fragment was purified from agarose gel with a QIAquide Gel Extraction kit (QIAGEN, Valencia, Calif.) and ligated with BgIII linker. After BgIII digestion, the scFv fragment was cloned into pAvCvSv vector (gift of Babie Teng, Institute of Molecular Medicine, University of Texas, Houston) through insertion at the BgIII restriction site. The resulting plasmid, designated pAd-scFv_{Hu-901}, with the scFv fragment inserted at the correct orientation with respect to the hCMV promoter contained in the vector, was selected based on restriction mapping analysis (FIG. 1; first schematic shown).

### EXAMPLE 2

### Preparation of DNA Construct for the Expression of Intact Anti-IgE with Humanized Variable Regions and Human Constant Regions.

DNA constructs for the expression of an intact humanized anti-IgE antibody was prepared as follows. Full length cDNA for the heavy and light chains of a humanized anti-IgE, Hu-901, was obtained by RT PCR. Total RNA was obtained from the Hu-901 cell line using TRIZOL reagent (Gibco) according to the manufacturer's instruction. 5 ml TRIZOL reagent was directly added into a 7.5 cm diameter culture dish to lyse the cells. The cell lysis step was followed by phase separation, RNA precipitation, and RNA wash steps. One tenth of the recovered RNA was used to generate total polyA+ cDNA, from which Hu-901 cDNA was amplified with the SerperScript Preamplification System for First Strand cDNA synthesis (Gibco) according to the manufacturer's instruction. One tenth of the synthesized total cDNA was used as a template to amplify Hu-901 cDNA with oligonucleotides:

| | | |
|---|---|---|
| 901VH5B | 5′GGAGATCTCCACAGTCCCTGAACACAC | (SEQ ID NO: 14); |
| 901CH3 | 5′TCATTTACCCGGAGACAGGGA | (SEQ ID NO: 15); and |
| 901CK3 | 5′CTAACACTCTCCCCTGTTGAA | (SEQ ID NO: 16). |

Expand High Fidelity PCR system (Boehringer Mannheim) was used to decrease the mistake. The final PCR product was cloned into the ZERO-BLUNT cloning vector and the sequences was verified by DNA sequencing. Both heavy and light chains were cloned into plasmid pAdTrack-CMV (Johns Hopkins University) with the incorporation of the EF-1α promoter for the light chain.

### EXAMPLE 3

### DNA Construct for the Expression of an Intact Anti-IgE Antibody with Humanized Variable Regions and Murine Constant Regions.

The DNA fragment encoding the variable region of humanized anti-IgE, Hu-901, heavy chain was obtained by PCR amplification using plasmid pHCMV-V_{H}-HCγ1 as the template and oligonucleotides:

| | | |
|---|---|---|
| H3-5BH | (SEQ ID NO:11); and | |
| H3-3BL | (5′TGAGCTCACGGTCACCAGGGT 3′) | (SEQ ID NO:21) |

as primers under the reaction conditions of 94° C., 1 min.; 55° C., 2 min.; and 72° C., 1 min. for 25 cycles. The PCR product was treated with Hind III followed by Klenow fragment and cloned into the Eco47 III and Hind III treated with pCDNA3/Vh_{MAM4.20}-mCγ2a plasmid and a subsequent removal of Vh_{MAM4.20} fragment. The resulting plasmid with Hu-901 V_{H} gene inserted at the correct orientation, pCDNA3/ Vh₉₀₁-mCγ2a, was purified and analyzed by an ABI Prism™ 300 Genetic Analyzer to confirm the Vh DNA sequence. The DNA fragment encoding Vh₉₀₁-mCγ2a was then obtained by Hind III and Not I double digestion of pCDNA3/ Vh₉₀₁-mCγ2a, followed by Klenow treatment, and cloned into pAvCvSv vector digested with BgI II, followed by treatments with Klenow and calf intestine alkaline phosphatase (CIAP). The resulting plasmid, designated pAdH901, contains Vh₉₀₁-mCγ2a placed under the promoter control of hCMV provided by the pAvCvSv vector.

The DNA fragment encoding the variable region of humanized anti-IgE, Hu-901, light chain was obtained by PCR amplification using plasmid pHCMV-V_{L}-HCκ as the template and oligonucleotides:

| | | |
|---|---|---|
| L1-5H | 5′TGAAGAAAGCTTGCCGCCACCATGGAG3′ | (SEQ ID NO:17); and |
| L1-3B | 5′GCATCCGCTCGTTTGATCTCCACCTTGGT3′ | (SEQ ID NO:18) |

as primers under the reaction conditions of 94° C., 1 min.; 55° C., 2 min.; and 72° C., 1 min., for 25 cycles. The DNA fragment encoding the constant region of murine Cκ chain was obtained by PCR amplification using plasmid pCDNA3/ Vh_{MAM4.20}-mCκ as the template and oligonucleotides:

| | | |
|---|---|---|
| MuK5-EBC | 5′ CGGAATTCGAGCGGATGC-TGCACCAACTGTATCGATCT 3′ | (SEQ ID NO:19); and |
| Muk3-x | 5′ GCTCTAGAGCTAAC-ACTCATTCCTGTTGAAGCTCTTGACA 3′ | (SEQ ID NO:20) |

as primers under the reaction conditions of 94° C., 1 min.; 55° C., 2 min.; and 72° C., 1 min. for 25 cycles. The VL-901 PCR product was digested with Bsr BI, purified after agarose gel electrophoresis, then ligated to mCκ with prior treatment with Bsr BI and XbaI. The ligated DNA was then subjected to PCR amplification using oligonucleotide primers L1-5H and Muk3-x (SEQ ID NO:20). The PCR product then was cloned into pCR® Blunt vector, and resulting plasmid pCR-VL₉₀₁-mCκ was analyzed by an ABI Prism™ 300 Genetic Analyzer to confirm the DNA sequence.

To place the VL₉₀₁-mCκ fragment under hCMV promoter control and eventually joined it with pAdH901 (mCγ2a) for the ultimate expression of the intact antibody, an intermediate holding vector was constructed. The DNA fragment containing hCMV promoter and enhancer sequences was obtained from pHCMV-V_{H}-HCγ2a by Cla I and Hind III digestion and cloned into pBluscript KS, previously digested with the same enzymes. The resulting plasmid was digested with Bam HI, treated with Klenow and CIAP, and used as the vector for the cloning of VL₉₀₁-mCκ fragment, which was obtained by Eco RI and Bam HI digestion of PCR-VL₉₀₁mCκ followed by Klenow treatment, to generate pKS-hCMV-L₉₀₁. A DNA fragment containing SV40 polyadenylation site was obtained by Hind III and Xba I digestions of plasmid pREP8 followed by Klenow treatment and then cloned into pKS-hCMV-L_{901(mCκ)} that was previously treated with Cla I, Klenow and CIAP, to generate pSpA-hCMV-L_{901(mCκ)}.

To create the final plasmid construct for the expression of Hu-901(mCγ2a,κ), plasmid pSpA-hCMV-L_{901(mCκ)} was digested with Not I, and the DNA fragment for SV40pA-hCMV-L_{901(mCκ)} was purified from agarose gel and cloned into pAdH901(mCγ2a), which was previously digested with Cla I, treated with Klenow, CIAP, ligated with Not I linker and subsequently digested with Not I. The resulting plasmid, pAdHu-901(mCγ2a,κ), contained heavy and light chain sequence of the humanized V/murine C antibody genes, each placed under independent hCMV promoter control and with its own polyadenylation signal downstream from the coding sequence.

### EXAMPLE 4

### Expression of Anti-IgE and Its scFv Fragment in Mammalian Cells via DNA Transfection.

The DNAs from plasmid pAd-scFv_{Hu-901} and pAdHu-901(mCγ2a,κ) were purified with NucleoBond® plasmid purification column (Clontech Laboratories, Inc. Palo Alto, Calif.), and used to transfect 293 cells (human embryo kidney epithelial cells; transformed with adenovirus 5 DNA) via electroporation (Gene Pulser™, BioRad Laboratories, Inc. Richmond, Calif.) under the following conditions: cell density, 10⁷ cells/ml containing 10 μg DNA in PBS, at 230 volts and 960 μF. After 10 minutes incubation at room temperature, the cells were placed in 60-mm dish containing 5 ml of EMEM medium with 10% fetal calf serum and cultured at 37° C. The culture supernatants were collected 4 days post transfection and the level of scFv_{Hu-901} and Hu-901 (mCγ2a,κ) were measured by ELISA. The scFv_{Hu-901} expression was measured by a competitive ELISA in which the wells of Immulon II plate (Dynatech Laboratories, Chantilly, Va.) were coated with goat anti-IgE (Jackson ImmunoResearch Laboratories, Inc., West Grove, Pa.) at 1 μg/ml for 16 hours at room temperature. The wells were then blocked with BLOTTO (5% not-fat milk in Phosphate buffered saline, 0.1% TWEEN and 0.01% Thimerosal) at room temperature for 2 hours. After being washed with PBST (PBS with 0.1% TWEEN 20), wells were reacted with a murine V/human Cε chimeric IgE, SE44 (Sun LK et al, Transfectomas expressing both secreted and membrane-bound forms of chimeric IgE with anti-viral specificity, 1991, J. Immunol. 146:199), at 0.5 μg/ml for one hour. Cultural supernatant was serially diluted at 1:2 and each dilution was mixed with equal volume of Hu-901-HRP conjugate at 1:16,000 dilution. One hundred microliters of the mixtures were then added to the washed wells and incubated for one hour at room temperature. After being washed, peroxidase substrate solution containing 0.1% 3′,3′,5′,5′-tetramethyl benzidine (Sigma Chemicals, St Louis Miss.) and 0.003% hydrogen peroxide (Sigma) was added at 200 μl/well and incubated for 30 minutes at room temperature. The reaction was stopped by the addition of 50 μl of 0.2 M sulfuric acid and the OD of the reaction mixture in each well was read with a BioTek ELISA reader (Winooski, Vt.). To determine the concentration of scFv_{Hu-901} produced by the transfected cells, purified Hu-901 was used to generate a standard curve. Using this assay, the cultural supernatant of 293 cells transfected with pAd-scFv_{Hu-901} was measured to contain approximately 4 μg/ml of scFv_{Hu-901} 4 days post transfection.

The Hu-901 (mCγ2a,κ) expression was measured by an ELISA in which the wells of Immulon II plate were coated with an anti-idiotypic antibody against Hu-901 (mAb69-76-5, Tanox proprietary antibody) at 1 μg/ml for 16 hours at room temperature. After the wells were blocked for 2 hours at room temperature, cultural supernatants from cells transfected with pAd-Hu-901 (mCγ2a,κ) at 1:2 serial dilutions were added to the wells at 50 μl/well and incubated for one hour at room temperature. After being washed, the wells were then added with 50 μl/well of mAb69-76-5-HRP conjugate at 1:1000 dilution, and incubated at room temperature for one hour. Afterwards, the wells were washed and peroxidase substrate solution was added at 100 μl/well and incubated for 30 minutes at room temperature. The reaction was stopped by the addition of 50 μl of 0.2 M sulfuric acid and the OD of the reaction mixture in each well was read with a BioTek ELISA reader (Winooski, Vt.). To determine the concentration of Hu-901 (mCγ2a,κ) produced by the transfected cells, purified Hu-901 was used to generate a standard curve. Using this assay, the cultural supernatant of 293 cells transfected with pAd-Hu-901(mCγ2a,κ) was measured to contain approximately 100 ng/ml of Hu-901 (mCγ2a,κ) 4 days post transfection.

### EXAMPLE 5

### Preparation of Recombinant Adenovirus Constructs for the Delivery of Anti-IgE Genes and Genes of Its scFv Fragment.

To change the mode of gene delivery into suitable hosts, plasmid constructs pAd-Hu-901 (mCγ2a,κ) and pAd-scFv_{Hu-901} can be incorporated into adenovirus genome and packaged into infectious viral particles. This is achieved by the inverted terminal repeat sequences contained in the plasmids for the packaging and a short segment of adenoviral genome that allows homologous recombination with a near full length adenoviral DNA between the overlapping regions. pAd-scFv_{Hu-901}, 10 μg, was mixed with 2 μg of plasmid pJM17 (McGrory W J, D S Bautista and F L Graham (1988) Virology 163:614-617.), which contained the adenovirus genome with DNA insertion at the El region. The DNA mixture was then used to co-transfect 293 cells using calcium phosphate transfection system (Life Technologies, Gaithersburg, Md.), in a 60-mm cultural dish. After being exposed to DNA, the culture was incubated in IMEM medium containing 10% FBS for overnight and then replaced with 5 ml tissue culture overlay agar (IMEM plus 1% SeaPlaque agarose). The second overlay was placed 4-5 days later. Adenovirus plaques appeared approximately 10-14 days post transfection. Isolated plaques were picked with long-stem pipette and the virus particles were recovered by repeated freeze/thaw cycles. The virus was then used to infect 293 cells at 24-well plates, and the cultures were harvested when cytopathic effect of the virus infection was apparent (approximately 3-5 days). One hundred microliters of the virus suspension was heat inactivated, and 10 μl of which from each plaque was subjected to PCR analysis using oligonucleotides H3-5BH and L1-3B as primers to determine whether the virus contain scFv_{Hu-901} gene. One isolated virus suspension that scored positive in this PCR analysis was further expanded by infection to 293 cells to generate crude virus lysate. To prepare highly purified virus stocks, 24 150-mm plates of 293 culture at approximately 80% confluence was infected with 80 μl of crude virus lysate in 2 ml of infection media per plate (IMEM containing 2% FBS) for 90 minutes with rocking at 37° C. Afterwards, 20 ml of IMEM medium containing 10% FBS was added to each plate and the cultures were incubated at 37° C. Thirty-six to forty-eight hours post infection when cytopathic effects were apparent, culture supernatant was aspirated and the cells were scraped off the plate with rubber policeman. Cell pellet were pooled and subjected with freeze/thaw cycles 4 times, and cell debris was removed by centrifugation at 10,000× g. The cleared virus suspension was then loaded to CsCl step gradients containing CsCl at 1.25 g/ml (3 ml) and 1.40 g/ml (3 ml) in each of a 12-ml nitrocellulose ultracentrifuge tube, and subjected to ultracentrifugation at 35 K rpm in a Sorvall AH41 rotor. The virus particles trapped in the interphase of the density gradient were collected, transferred to a second tube containing CsCl at 1.33 g/ml and centrifuged at 35 K for 24 hours. The double-banded virus particles were collected from gradient, dialyzed against TMG buffer containing 10 mM Tris, pH 7.4, 1 mM MgCl₂, and 10% (v/v) glycerol with 3-4 changes of buffer. The virus preparation thus obtained was distributed in small aliquots and stored at −70° C. The infectious titer of the virus stock was determined to be approximately 1-2×10¹⁰ plaque forming unit per ml using standard titration method.

To generate the virus construct containing Hu-901 (mCγ2a,κ) genes, an additional method was used. This method allows homologous recombination to occur in E. coli for Hu-901 (mCγ2a,κ) genes to incorporated into viral genome as described. This was accomplished by transfer of the Hu-901 (mCγ2a,κ) genes into pAd-Shuttle-CMV vector (He, T -C, S Zhou, L T da Costa, J Yu, K W Kinzler and B Vogelstein (1998) Proc. Natl. Acad. Sci. USA 95:2509-2514) by stepwise insertion through the Not I site of the vector. The resulting plasmid, pAd-Shuttle-Hu-901 (mCγ2a,κ) (FIG. 1, third schematic shown), was then used along with pAdeasy-1 to cotransform E. coli BJ5183. Kanamycin-resistant transformants were analyzed by restriction analysis to identify clones undergone the recombination, resulting in the incorporation of Hu-901(mCγ2a,κ) into viral genome. The plasmid DNA was purified, and used to transfect 293 cells via electroporation. Culture supernatant was collected 10 days post transfection, and shown to contain Hu-901(mCγ2a,κ) using the ELISA method described in Example 2. The cultural supernatant was then used to expand and preparation for Ad-Hu-901(mCγ2a,κ) virus stock using procedures as described above. The virus stock thus obtained was determined to contain approximately 1×10¹⁰ pfu/ml. FIG. 1 shows the schematic diagrams of the recombinant adenoviral constructs.

### Example 6

### Expression of Anti-IgE and scFv in Recombinant Adenovirus Infected Cells.

Crude virus lysates of Ad-Hu-901 (mCγ2a,κ) and Ad-scFv_{Hu-901} viruses were used to infect 293 cells at a multiplicity of infection of 1 in 100-mm cultural dish. Four days post infection, cultural supernatants were collected, cleared of cell debris, and assayed for the expression of Hu-901 (mCγ2a,κ) and scFv_{Hu-901} using ELISA procedures as ones described in Example 2, it was determined that the cultural supernatant contained 1 μg/ml and 8 μg/ml of Hu-901(mCγ2a,κ) and scFv_{Hu-901}, respectively. These assays also demonstrated the binding ability of the expressed protein, i.e., Hu-901(mCγ2a,κ) to its anti-idiotypic antibody mAb 69-76-5, and scFv_{Hu-901} for its ability to compete with intact Hu-901 antibody in binding IgE. With slight modification of the assay format in which the goat anti-mouse IgG2a-HRP conjugate was used as the tracer antibody, it was also demonstrated that the expressed intact antibody exhibited murine IgG2a isotype. The expressed scFv_{Hu-901} was further analyzed by Western blot analysis. Twenty microliters of supernatant from Ad-scFv_{Hu-901} viruses-infected cultural were resolved in 10% SDS-polyacrylamide gel under either reducing conditions, and the proteins were transblotted onto nitrocellulose membrane. The membrane was then incubated in BLOTTO buffer for 1 hour at room temperature to block excess protein binding sites. It was subsequently reacted with mAb 69-76-5 at room temperature for overnight, followed by goat anti-mouse IgG Fc-HRP conjugate, at room temperature for one hour. In between the antibody incubations, the membrane was washed 3 times, 5 minutes each, with PBST. After final wash, the membranes were reacted with one component TMB membrane peroxidase substrate solution (kirkegaard & Perry Laboratory, Gaithersburg, Md.). A protein band consistent with the size for a scFv protein was shown in a gel. The same gel showed the band pattern of affinity purified scFv_{Hu-901} for comparison.

### EXAMPLE 7

### Expression of Anti-IgE in FVB Mice Infected with Recombinant Adenovirus Constructs.

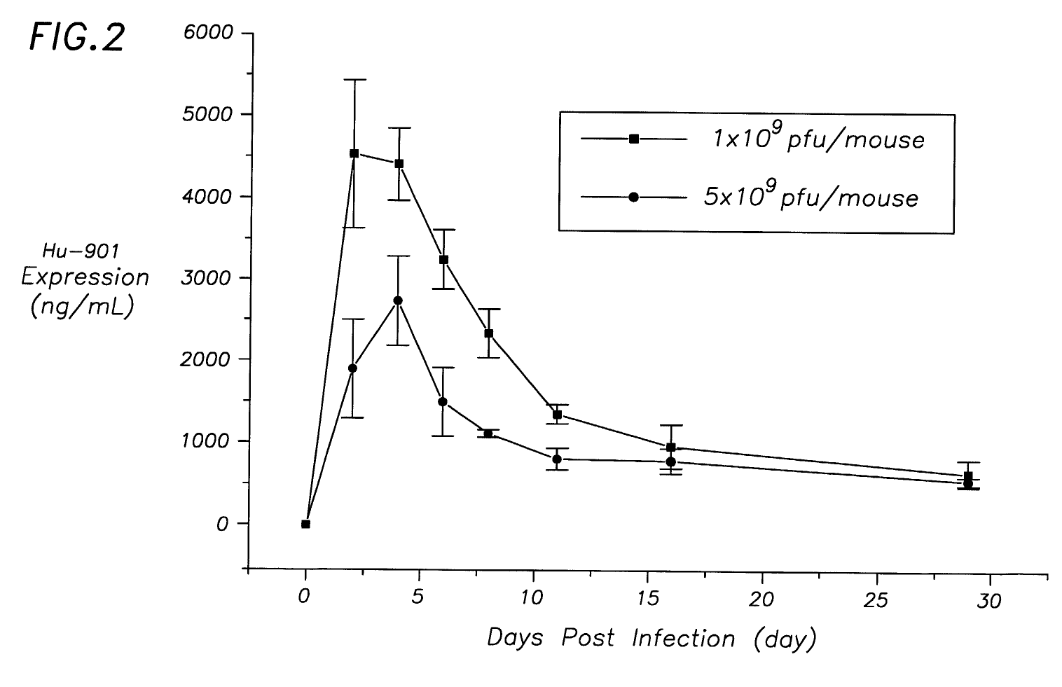

Purified virus particles were used to infect two groups of FVB mice through tail vein. The amount of AdHu-901 (mCγ2a,κ) virus were 5×10⁸ and 1×10⁹ pfu/mouse. Serum samples from treated animals were collected on day 1 prior to injection and on days 2, 4, 6, 8, 11, 16, 29, and 46 post injection. Expression of scFv was measured by an ELISA as described in Example 2. In that assay, mAb67-76-5 was immobilized onto wells of Immunlon II plates to capture the expressed Hu-901 (mCγ2a,κ), and the captured antibodies were detected by the mAb69-76-5-HRP conjugate followed by color development of the enzyme substrate. Purified Hu-901 served as standard for the quantitative assay. Results displayed in FIG. 2 showed a dose-dependent expression of the active antibody, which peaked on days 2-4 post infection with the serum concentration reaching approximately 4.5 μg/ml and 2.5 μg/ml, for mice receiving 1×10⁹ and 5×10⁸ pfu/mouse of virus, respectively. For infection dose at 5×10⁹ pfu/mouse, the side effects of the virus infection proved to be too toxic, and mice died within 4 days post infection. The serum levels of the expressed Hu-901 (mCγ2a,κ) antibody quickly decreased to about 20-35% of peak level on day 11, and remained at approximately that level to day 46.

### EXAMPLE 8

### Expression of scFv in FVB Mice Infected with Recombinant Adenovirus Constructs.

Purified virus particles were used to infect 6 FVB mice through tail vein. The amount of Ad scFv _{Hu901} virus ranged from 5×10⁸ (2 mice), 1×10⁹ (2 mice), 2.5×10⁹ (1 mouse) and 5×10⁹ (1 mouse) pfu/mouse. Serum samples from treated animals were collected on day 1 prior to injection and on days 2, 4, 6, 8, 11, 15, 21, and 28, 35 and 58 post injection. Expression of scFv was measured by a competitive ELISA as described in Example 2. In that assay, wells of Immunlon II plates were immobilized with goat-anti-IgE to capture a monoclonal IgE and the expressed scFv was quantified by the ability in competing with purified scFv protein for binding to IgE.

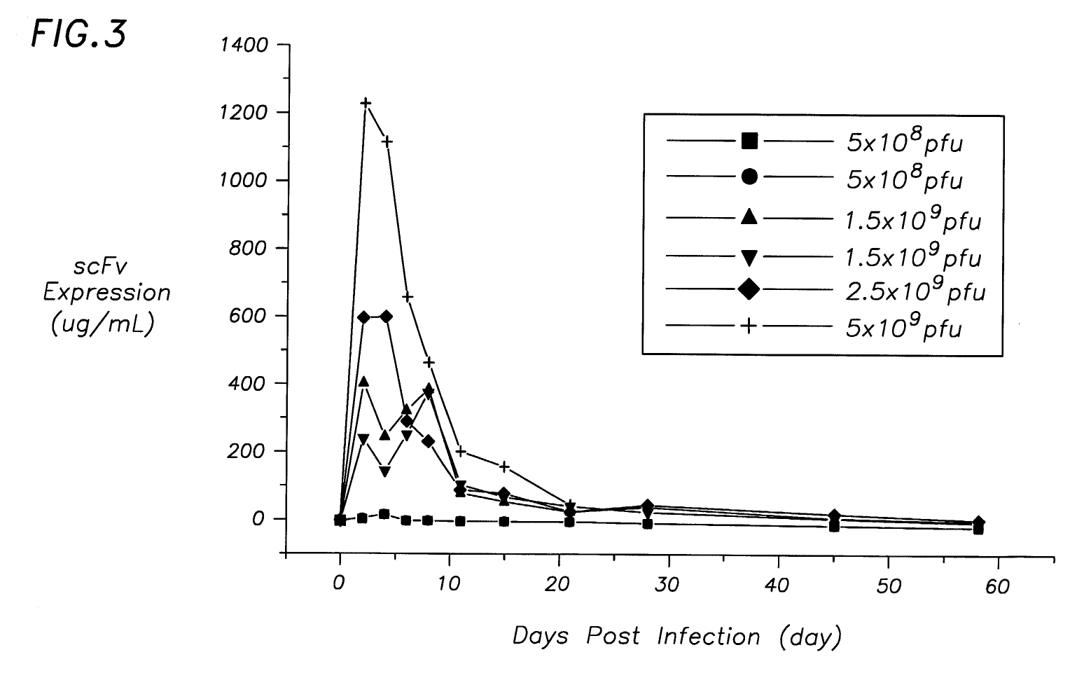

Results shown in FIG. 3 indicated a dose-dependent expression of scFv _{Hu901} in infected animals. At higher doses of infection tested (2.5×10⁹ and 5×10⁹ pfu/mouse), peak expression occurred on day 2 post infection, quickly decreased afterwards to essentially a residual level of expression beyond day 21 (less than 40 μg/ml). In the next dose tested (1×10⁹ pfu/mouse), peak expression appeared to occur on day 6 or later and quickly decreased to residual expression. In the lowest dose tested (5×10⁸ pfu/mouse), only low but appreciable levels of scFv expression was observed throughout the experiment. To examine the expressed scFv protein, 5 μl of serum from each infected animal collected on day 2 were resolved in 10% SDS-PAGE, transblotted onto nitrocellulose membrane and reacted to mAb 69-76-5, followed by goat anti-mouse IgG (Fc)-HRP conjugate. The result of this immunoblot analysis indicated that the scFv expressed in the serum of animals infected with the lowest dose of recombinant virus (5×10⁸ pfu/mouse) was barely detectable (lanes 1 and 2), whereas in sera of animals infected with higher doses of virus, scFv expression was evident, giving bands consistent with molecular weight expected for scFv.

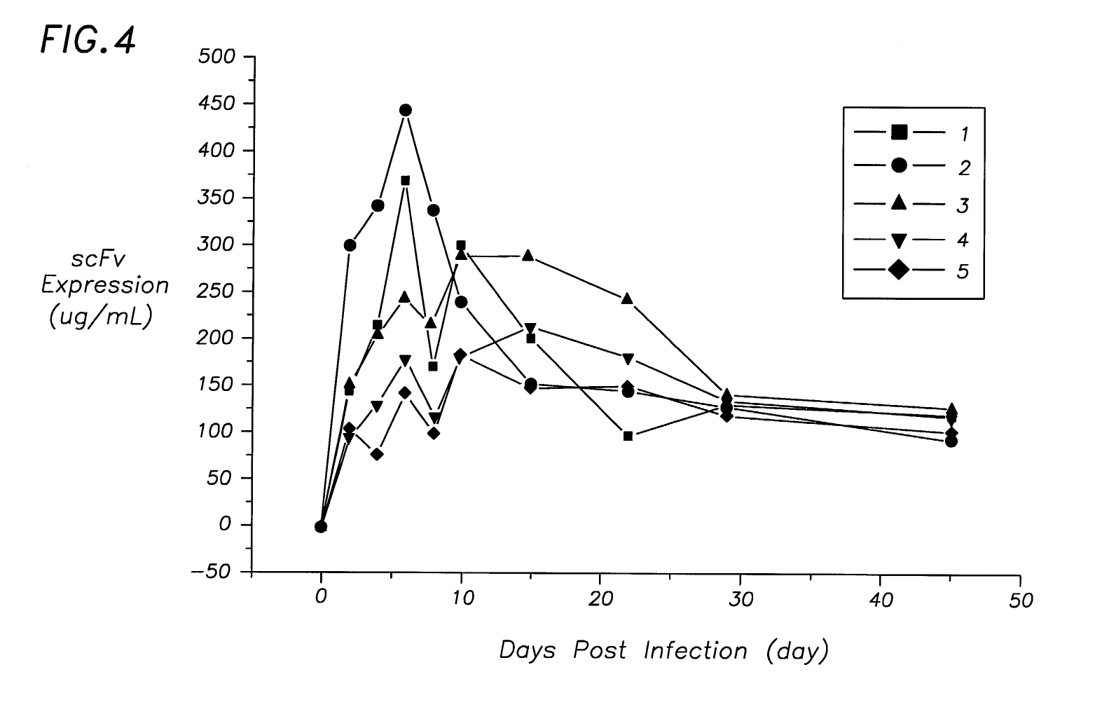

To test the variation of scFv expression in different mouse receiving the same dose of recombinant adenovirus, 5 FVB mice were infected with scFv_{Hu901} at 1.5×10⁹ pfu/mouse. Serum samples from infected mice were collected on appropriate days post infection and measured for scFv expression by competitive ELISA described previously. Results shown in FIG. 4 suggested that different host animal responded differently to the virus infection and exhibited different levels of scFv expression. Although it was unlikely, it could not be totally ruled out that these mice did not received equal amount of virus during injection. It is also worthy to note that in this experiment, all mice still exhibited significant levels of scFv expression on day 45 (100 μg/ml), whereas in an earlier pilot experiment, 2 mice receiving presumably the same dose of virus showed lower level of scFv expression (50 μg/ml, see FIG. 2). This difference may reflect the difference in exact virus particles administered to the animals since they were from two different batches of preparation.

### EXAMPLE 9

### Immune Response to the Expressed scFv.

Host response to the expressed transgene product, i.e., anti-Hu-901(scFv) antibody response in the virus infected mice, was initially measured in an ELISA which detects the antibody reactivity to V regions of Hu-901. In this assay, wells of Immunlon II plates were coated with Hu-901 antibody. Serum samples of infected mice at 1:10 dilution were added to these wells and incubated for one hour at room temperature. After non-reactive materials were washed off, the immune complex was detected by HRP-conjugated Hu-901, followed by color development of enzyme substrate.

Under these assay conditions the results indicate that only mice infected with low dose of virus (5×10⁸ pfu/mouse) exhibited detectable levels of anti-scFv_{Hu-901} response, whereas anti-scFv_{Hu-901} response in mice infected with higher doses of virus was not detectable even on day 58. However, while this assay can directly measure the anti-scFv_{Hu-901} or anti-Hu-901(mCγ2a,κ) antibodies, it cannot detect these responses when the antibodies are complexed with excess of expressed scFv_{Hu-901} or Hu-901 (mCγ2a,κ) in serum.

To test whether animals infected with higher doses of AdscFv_{Hu-901}virus indeed induced immune responses, an alternative assay was used. In this assay, wells of Immulon II plates were immobilized with goat anti-human K antibody to catch the immune complexes through binding to scFv_{Hu-901} in serum samples. The complexes adsorbed onto the wells were then detected with HRP conjugate of goat anti-mouse Fc antibody. Under these assay conditions all serum samples of AdscFv_{Hu-901} virus-infected mice were shown to have anti-antibody responses post day 8 of infection. However, because of the lack of appropriate immune complexes to serve as standard solutions, and also because sample dilution was needed for the assay, which tended to cause partial dissociation of the complexes and obscured an accurate determination of the complexes, this assay did not allow a quantitative measurement of the level of the immune responses in these animals. This assay could not measure the antibody responses in mice infected with AdHu-901 (mCγ2a,κ) virus, since the expressed Hu-901(mCγ2a,κ) antibody contained murine constant regions and would bind to tracer antibodies in the assay even if it was bound to the wells devoid of anti-antibodies attached to it.

### EXAMPLE 10

### Generation of a Transgenic Mouse Line that Expresses an IgE Antibody Containing Human Cs Sequence.

Chimeric Ig gene comprising human Cε region and the H chain V region of the murine Mab BAT123 (an anti-HIV antibody) was constructed. This chimeric gene was inserted into a pSV2gpt (L. K. Sun et al. J. Immunol. 146: 199-205, 1991) and the resulting plasmid was used as the ε transgene. Two hundred pg of the transgene plasmid DNA was microinjected into the nucleus of each egg from the FVB mice. A total of 128 fertilized eggs that survived pronuclear microinjections of the transgene were implanted in the oviduct of recipient female mice. From 23 offspring, three contained human Cε sequences. Genomic DNA was prepared from a 1-cm segment from the tail. Copy numbers of the human ε transgene per haploid genome were determined by quantitative slot blots using the transgene plasmid DNA as the standards. Serum IgE levels were determined by ELISA using purified BAT123IgE as standards. The results are shown in Table 1. These three founder mice were used to establish transgenic mouse lines. The properties of the F1, F2, and F3 mice are summarized in Tables 2 and 3. For experiments described below in Example 11, F2 or F3 transgenic mice expressing serum human IgE levels of 1 to 10 μg/ml were used.

TABLE 1
Characteristics of the founder transgenic mice.

| | Copy number of the ε | Serum human IgE level |
|---|---|---|
| Mouse | transgene per haploid | (μg/ml) |
| 21282 | 25 | 2.7 |
| 21288 | 25 | 5.8 |
| 21296 | 3 | 10.8 |

TABLE 2
Transgenic F1 and F2 mouse lines.

| | F1 | | | F2 | | |
|---|---|---|---|---|---|---|
| | | | Transgene | | | Transgene |
| | Serum human IgE¹ | | integration^{2, 3} | Serum human IgE¹ | | integration^{2, 3} |
| F0 | μg/ml | % positive | (%) | μg/ml | % positive | (%) |
| 21282 | 0.09-1.9 | 18/48⁴ (38) | +6/17 (35) | 0.01-6.8 | 33/42 (78) | ++4/19 (21) |
| | | | | | | +13/19 (68) |
| 21288 | 0.04-1.6 | 12/32 (38) | +3/8 (38) | 0.05-4.1 | 28/41 (68) | ++6/20 (30) |
| | | | | | | +10/20 (50) |
| 21296 | 0.02-5.9 | 18/33 (54) | +5/11 (45) | 0.01-3.7 | 24/31 (77) | ++6/31 (19) |
| | | | | | | +17/31 (55) |

¹Serum levels of human IgE were determined by ELISA with a detection limit of 0.01 mg/ml.
²Transgene integration was determined by quantitative slot blots.
³+, heterozygotes; ++, homozygotes.
⁴Number of positive mice/number of mice analyzed.

TABLE 3
Transgenic F3 mouse lines.

| | F3 | | | |
|---|---|---|---|---|
| | Serum human IgE¹ | | | Transgene integration^{2,3} |
| F0 | μg/ml | % positive | | (%) |
| 21282 | 0.02-3.5 | 11/11⁴ | (100) | ++11/11 (100) |
| 21288 | 0.15-2.7 | 21/21 | (100) | ++17/17 (100) |

¹Serum levels of human IgE were determined by ELISA with a detection limit of 0.01 mg/ml.
²Transgene integration was determined by quantitative slot blots.
³++, homozygotes.
⁴Number of positive mice/number of mice analyzed.

### EXAMPLE 11

### Suppression of Human Cs-Containing IgE in Serum of Transgenic Mice Infected with Recombinant Adenovirus

Heterozygous F1 progeny of the Hu-IgE transgenic mice, with circulating human Cε-containing IgE at a concentration in the range of 2-12 μg/ml, were used to test the ability of recombinant adenovirus constructs to suppress serum IgE. Two groups of mice, each consisting of 5 mice, were infected through tail vein injection of 1×10⁹ infectious units of AdscFv_{Hu901} and Ad-Hu-901 (mCγ2a,κ), respectively. Serum samples from treated animals were collected several times prior to injection and on days 2, 4, 6, 9, 16, and 28 post injection. Free serum human Cε-containing IgE in these samples was measured in an ELISA as follows. Wells of Immulon II plates were immobilized with mAb Hu-901 at 1.5 μg/ml at room temperature for overnight. The wells were then blocked with BLOTTO at room temperature for 2 hours. After being washed with PBST, 50 μl of serum samples diluted at 1:10 with BLOTTO were added to the wells and incubated at room temperature for one hour. After the unbound materials were washed off, the wells were reacted with 50 μl of mAb E-10-10-3 at 1.5 μg/ml at room temperature for one hour. Mab E-10-10-3 is an anti-IgE which binds to IgE at an epitope not overlapping with Hu-901. After washing, wells were then incubated with 50 μl of HRP conjugate of goat anti-mouse IgG Fc at 1:1000 dilution for one hour at room temperature. Finally, 100 μl of the peroxidase substrate solution were added after wash and incubated at room temperature for 30 minutes. The enzyme reaction was terminated with the addition of 50 μl of 0.2 M H₂SO₄, and the OD of the reaction mixture in each well was read at 450 nm. The working range of the assay was between 2 and 64 ng/ml.

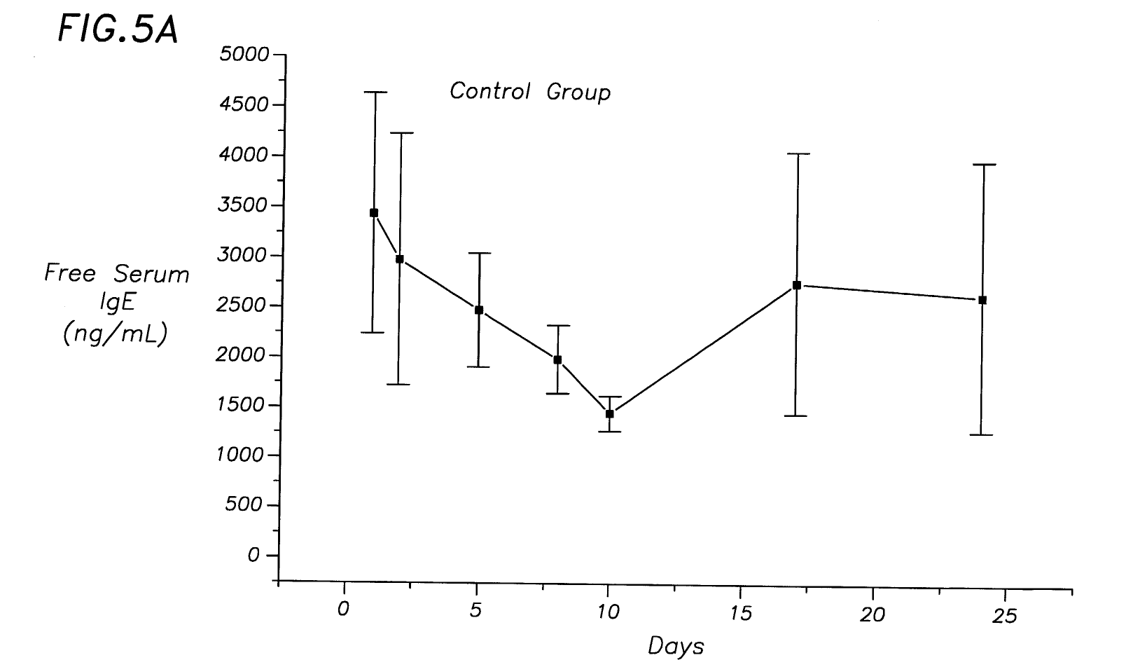

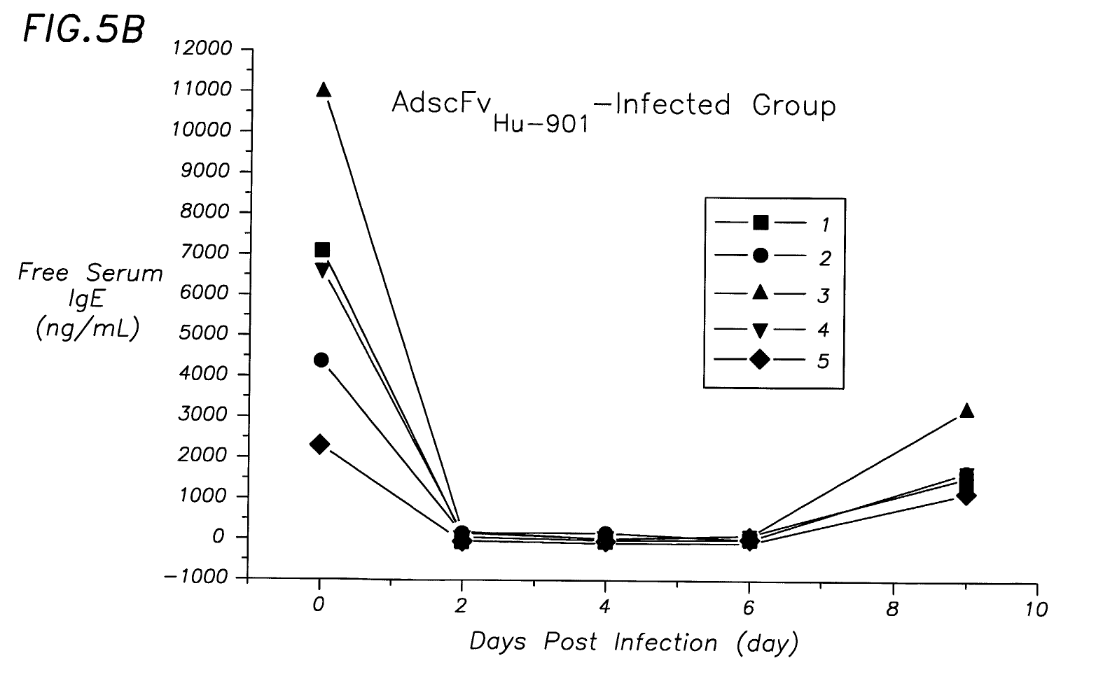

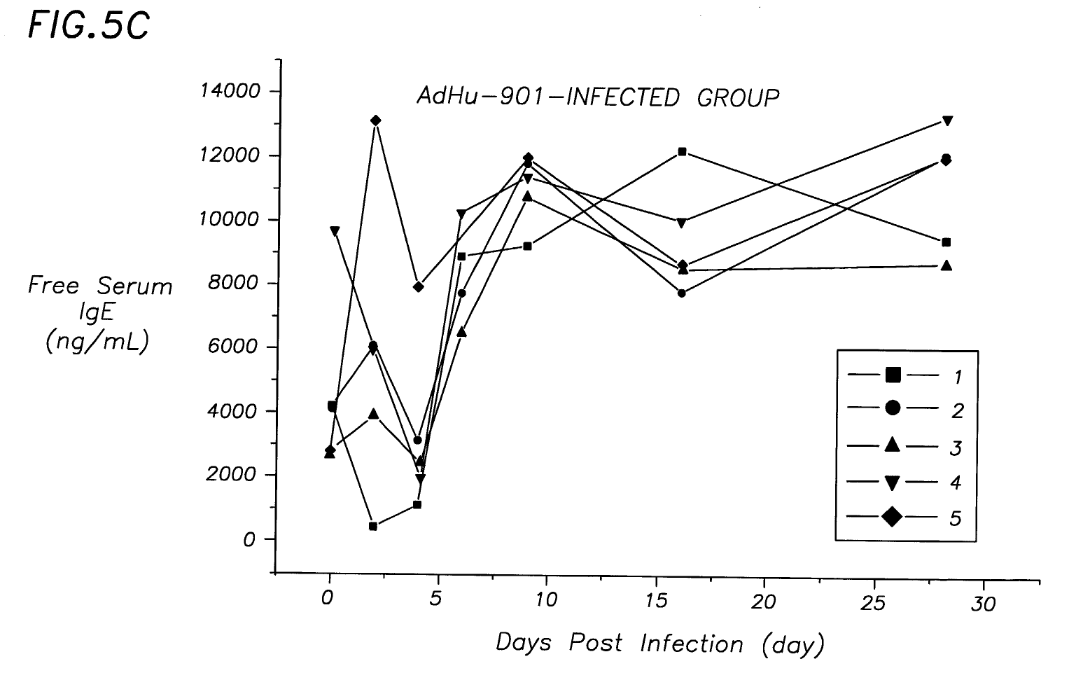

As shown in FIG. 5A, serum IgE of untreated mice fluctuated significantly during the course of this experiment. Under no circumstances, however, did this fluctuation result in the reduction of circulating IgE to less than 1.5 μg/ml. On the contrary, a sharp decrease in free serum IgE to a level between 20-200 ng/ml was noted in mice infected with AdscFv_{Hu901} (FIG. 5B), representing greater than 96% of the suppression of free circulating IgE in these animals. This was apparently due to the suppression by the expressed scFv _{Hu904} in these mice, in which great majority, if not all, of IgE was bound by scFv _{Hu901}. This suppression of circulating IgE lasted over 6 days, and IgE levels started to bounce back afterwards, coinciding with the decrease in the expression of scFv _{Hu901} in these mice. It has to be noted that the free IgE measured in this assay represented a slight over-estimation, since serum samples had to be diluted 10 fold, resulting in partial dissociation of the IgE-scFv _{Hu901} immune complex. It was also due to this dilution related dissociation, that free IgE levels in serum samples collected beyond day 9 could not be accurately measured, since these samples required higher dilution in order to bring IgE levels to within the working range of the assay.

Ad-Hu-901(mCγ2a,κ) was less effective in suppressing IgE in these transgenic mice. As noted in FIG. 5C, infection of Ad-Hu-901 (mCγ2a,κ) only resulted in a brief and less then complete suppression of IgE in these mice, achieving approximately 40-90% of IgE suppression only on day 4 post infection. This was perhaps due to a much lower level of expression of intact Hu-901 (mCγ2a,κ) in the infected mice (FIG. 2).

Overall, this experiment demonstrated that in vivo delivery of scFv _{Hu901} gene via a recombinant adenoviral vector could result in a high level expression of scFv _{Hu901}. The expressed scFv _{Hu901} subsequently bound circulating IgE, resulting in a drastic reduction of free IgE for a period of time. This approach should provide an alternative approach to deliver anti-IgE or its antibody fragments for therapeutic application of IgE-mediated allergic diseases.

It should be understood that the foregoing description and examples are descriptive only and not limiting, and that the scope of the invention is limited only by the claims which follow, and includes all equivalents of the subject matter of the claims.

What is claimed is:

1. A method of expressing an anti-IgE antibody, or a functional variant or fragment thereof, which does not bind to IgE bound to FcεRI, in a host cell comprising introducing an expression vector encoding the anti-IgE antibody, or functional variant or fragment thereof, into a host cell and maintaining the expression in the cell.

2. The method of claim 1, wherein the antibody, or functional variant or fragment thereof, inhibits binding of IgE to FcεRI, FcεRII, or to both receptors.

3. The method of claim 1 or claim 2, wherein the expression vector encodes an anti-IgE antibody Hu-901, having the Accession No. ATCC 11130.

4. The method of claim 1 or claim 2, wherein the nucleic acid encodes an scFv fragment of the anti-IgE antibody Hu-901, having the Accession No. ATCC 11130.

5. A method of inducing a host cell to express an anti-IgE antibody, or a functional variant or fragment thereof, comprising administering an expression vector encoding an anti-IgE antibody, or a functional variant or fragment thereof.

6. The method of claim 5, wherein the antibody, or functional variant or fragment thereof, inhibits binding of IgE to FcεRI, FcεRII, or to both receptors.

7. The method of claim 5 or claim 6, wherein the expression vector encodes an anti-IgE antibody Hu-901, having the Accession No. ATCC 11130.

8. The method of claim 5 or claim 6, wherein the nucleic acid encodes an scFv fragment of anti-IgE antibody Hu-901.

9. A method of expressing in a host cell an anti-IgE antibody, or a functional variant or fragment thereof, comprising administering a formualtion comprising a nucleic acid sequence encoding an anti-IgE antibody or a functional variant or fragment thereof, which does not bind to IgE bound to FcεRI.

10. The method of claim 9, wherein the antibody, or functional variant or fragment thereof, inhibits binding of IgE to FcεRI, FcεRII, or to both receptors.

11. The method of claim 9 or claim 10, wherein the nucleic acid encodes an anti-IgE antibody Hu-901, having the Accession No. ATCC 11130.

12. The method of claim 9 or claim 10, wherein the nucleic acid encodes an scFv fragment of the anti-IgE antibody Hu-901, having the Accession No. ATCC 11130.

13. A method of treating an IgE-mediated allergic disease comprising administering a formulation comprising an expression vector encoding an anti-IgE antibody, or functional variant or fragment thereof, which does not bind to IgE bound to FcεRI.

14. The method of claim 13, wherein the antibody, or functional variant or fragment thereof, inhibits binding of IgE to FcεRI, FcεRII, or to both receptors.

15. The method of claim 13 or claim 14, wherein the expression vector encodes an anti-IgE antibody Hu-901, having the Accession No. ATCC 11130.

16. The method of claim 13 or claim 14, wherein the nucleic acid encodes an scFv fragment of the anti-IgE antibody Hu-901, having the Accession No. ATCC 11130.
